(19) United States
(12) Patent Application Publication
(10) Pub. No.: US 2015/0202353 A1
(43) Pub. Date: Jul. 23, 2015

(54) Flexible Exudate Collection Canister

(71) Applicant: Christopher John Daughtery, Monroe, NJ (US)

(72) Inventor: Christopher John Daughtery, Monroe, NJ (US)

(21) Appl. No.: 14/160,754

(22) Filed: Jan. 22, 2014

(51) Int. Cl. A61M 1/00

(52) U.S. Cl. CPC A61M 1/0001; A61M 1/0088

(57) ABSTRACT

An orientation independent, disposable, flexible exudate management canister that is capable of conforming to curved surfaces, for collecting exudate fluids from a wound as part of a negative pressure wound therapy system. The canister may be used with existing Negative Pressure Wound Therapy (NPWT) suction pumps and wound dressings, or with the suction pump and wound dressing described in this embodiment to make a complete NPWT system.

### CROSS-REFERENCED TO RELATED APPLICATIONS

Provisional application No. 61/755,534, filed on 23 Jan. 2013.

### FEDERALLY SPONSORED RESEARCH

Not applicable

### SEQUENCE LISTING

Not applicable

### FIELD OF THE INVENTION

This invention relates to systems for treating wounds by the application of negative pressure, and to devices for use in such systems.

### BACKGROUND

It is well known that wounds can be treated by applying suction to the wounds under an airtight wound cover to aspirate wound exudate and other liquids from the wound, lowering the bacterial bio-burden and promoting wound healing.

There are numerous system available, with varying features, based around creating a vacuum with a vacuum source, applying the vacuum to a wound site, applying a wound cover to seal the wound, providing a wound filler in the wound to prevent the wound cover being drawn into the wound, drawing the exudate from the wound under vacuum and storing the wound exudate in some kind of wound exudate canister. The vacuum source, exudate storage canister and wound cover are normally connected together with flexible tubing, but some components may be connected directly to others. U.S. Pat. No. 5,645,081 Argenta et al, describes the use of negative pressure wound therapy and proposes the use of open celled wound fillers. U.S. Pat. No. 7,216,651 Argenta et al, describes the use of a source of vacuum coupled to a vessel to contain the exudate flow which is in turn connected to a wound dressing.

Wounds with a large exudate flow (for example greater than 50 cc/day) often require a large vacuum source, usually an electrically driven pump and a high volume exudate canister, usually made from a rigid material. These systems are not easily portable and usually require the patient to remain in a fixed place during treatment. U.S. Pat. No. 7,857,806 Karpowski et al, describes a typical negative pressure wound therapy system comprising of an electrically driven pump and controller, rigid exudate canister and wound dressing for use in a medical facility.

Wounds with a small exudate flow (for example less than 50 cc/day) do not need a large vacuum pump or a large exudate canister, and so are suitable for treatment with a smaller, lighter and portable system. Many such portable negative pressure wound therapy systems exist on the market and are often used for patients who are in a homecare environment. These systems primarily fall into one of two categories, those that pull the wound exudate into an absorbent dressing and those that pull the exudate into a rigid canister attached to the pump.

Portable systems that use an absorbent dressing to contain the exudate have the disadvantage of making a bulky dressing at the wound site, increasing the risk of applying unwanted pressure to the wound, hold the exudate near to the wound which increases the risk of infection, prevents easy examination of the wound and the entire wound dressing needs to be changed when full of exudate, which can unnecessarily disturb the wound bed. U.S. Pat. No. 7,779,625 Joshi et al, describes such a system comprising of a small portable pump which pulls the exudate into a dressing which has an exudate storage system in the dressing.

Portable systems that use a rigid canister attached to the pump have the disadvantage of a bulky canister to be carried around that is the same size full or empty, and are orientation dependent to allow the exudate to fall into the canister, risking tips and spills in a portable product, and hold the exudate close to the pump. As the pump must be monitored frequently for correct operation and alarm activation, having the exudate canister attached to the pump risks embarrassment and loss of dignity to the user if wound exudate is then seen by others nearby. U.S. Pat. No. 8,257,328 Augustine et al, describes such a system and discusses the use of a ball float valve in the canister to prevent leakage from moving or tipping.

It would be advantageous to have a portable negative pressure wound system that has an exudate collection system that is orientation independent, flexible and could conform to curved body parts for easy carrying and is independent from the wound interface for easy changing without disturbing the wound bed. Such a system could hold the exudate away for the wound site to lower the risk of infection and could hold the exudate away from the pump so that the pump could be discretely worn by the user, without the risk of other people seeing the exudate. It would be further advantageous to hold the exudate without risk of leakage when the patient is moving about, and is as small and discreet as possible to maintain the patient's dignity.

U.S. Pat. No. 8,034,038 Biggie et al, describes the use of a portable negative pressure wound therapy system with a flexible disposable canister, filled with a superabsorbent polymer. However this invention is orientation dependent, hanging the canister from the pump body and requires an approximate vertical orientation of the canister to work as it has lower, middle and upper segments. The device described in this patent also requires a supporting structure inside the exudate collection bag to keep the flexible walls of the bag from collapsing in under the forces of the vacuum, increasing the empty size of the canister and limiting its ability to conform to curved surfaces, such as a patient limb.

This invention described here relates to the use of a flexible exudate collection canister as part of a portable Negative Pressure Wound Therapy (NPWT) system to overcome the disadvantages discussed above. The flexible exudate collection canister is made from flexible materials so that it can form itself to the shape of the human body and be worn discretely by a patient who is mobile and active in their daily lives. It has an exudate handling core made from a highly fluid absorbent material covered with an impermeable flexible outer cover, an air distribution layer, a first inlet for connecting it to the wound interface, a second inlet for connecting it to a pump and a hydrophobic membrane covering the input between the canister and the suction pump to prevent fluid from leaving the canister and moving towards the pump. The flexible exudate canister is separate from the wound dressing and the suction pump, and is connected to them via flexible plastic tubing. This allows the canister to be mounted discretely on the user at an optimum position and changed when full without disturbing the wound dressing. The canister does not need any internal support structure to stop its walls collapsing under the forces of the vacuum, so it can be manufactured as thin as the wall and fluid core will allow, and expand in thickness as it absorbs exudate until it reaches its fluid capacity. The flexible nature, orientation independence, ability to be mounted on a patient limb away from the pump, thin size and ease of changing without disturbing the wound be give this invention an advantage over the current devices.

### SUMMARY

This invention relates to the use of a flexible exudate collection canister as part of a portable Negative Pressure Wound Therapy (NPWT) system. The flexible exudate collection canister is made from flexible materials so that it can form itself to the shape of the human body and be worn discretely by a patient who is mobile and active in their daily lives. It has an exudate handling core made from a highly fluid absorbent material covered with an impermeable flexible outer cover, an air distribution layer, a first inlet for connecting it to the wound interface, a second inlet for connecting it to a pump and a hydrophobic membrane covering the input between the canister and the suction pump to prevent fluid from leaving the canister and moving towards the pump. The flexible exudate canister is separate from the wound dressing and the suction pump, and is connected to them via flexible plastic tubing. This allows the canister to be mounted discretely on the user at an optimum position and changed when full without disturbing the wound dressing.

### BRIEF DESCRIPTION OF THE DRAWINGS

FIG. 1 is a block diagram illustrating the components of a preferred embodiment of the negative pressure wound therapy system, showing the flexible exudate canister in relation to other components in the system.

FIG. 2 is a block diagram illustrating the components of a preferred embodiment of the suction pump.

FIG. 3 illustrates an exploded perspective view of a preferred embodiment of the flexible exudate canister.

FIG. 4 illustrates a partial exploded perspective view of a preferred embodiment of the flexible exudate canister showing the hydrophobic filter membrane and conduit connections.

FIG. 5 is a side cutaway view of a preferred embodiment of the wound dressing and wound interface.

### DETAILED DESCRIPTION

It will be readily understood that the components of the embodiment as generally described and illustrated in the Figures herein could be arranged and designed in a wide variety of different sizes, shapes and combinations. The following detailed description is not intended to limit the scope of the present disclosure, but merely representative of the preferred embodiment. The present invention may be embodied in other specific forms without departing from its spirit or essential characteristics. One skilled in the art will recognize that the invention can be practiced without one or more specific features, and that the features are not limited by their shape, size of combination. This invention relates to the use of an orientation independent, flexible exudate canister for use in a negative pressure wound therapy system, and as such may be used with other wound vacuum pumps or wound interfaces and wound dressings than that which is described here, but the detail has been limited in the description of those items to avoid obscuring aspects of the invention.

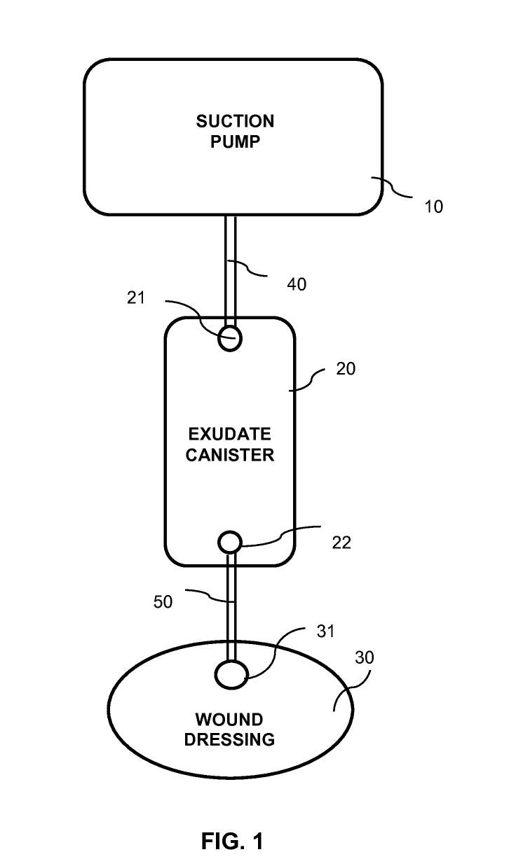

The proposed invention shown in FIG. 1 as a block diagram consists of an orientation independent, flexible exudate canister 20 with an input 21 to a suction pump 10 and an input 22 to a wound dressing 30. The flexible exudate canister 20 is connected by a flexible tube 40 to a portable suction pump 10 that will provide a source of vacuum to the canister 20. The flexible exudate canister 20 is connected to the wound dressing 30 by a second flexible tube 50. The wound dressing 30 consists of a wound interface connection 31 for the flexible tube 50 to a commercially available wound dressing 30. This allows the flexible exudate canister 20 to be positioned on a user's body away from the wound site in a place that is not loaded by the weight of the patient in everyday life, and allows the flexible exudate canister 20 to be changed when full without disturbing the wound bed.

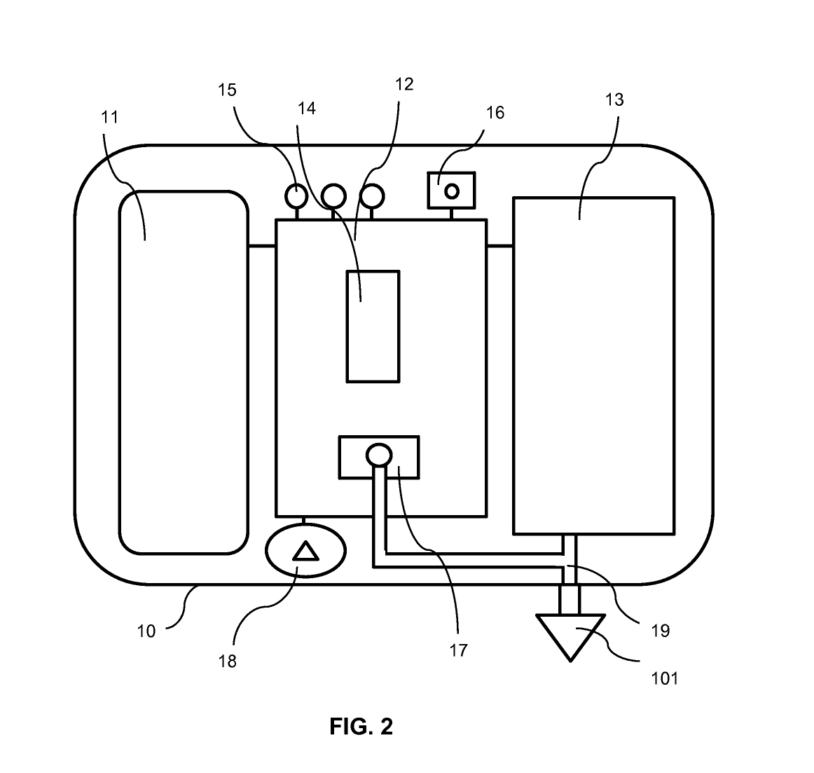

FIG. 2 shows the components of a portable suction pump suitable for use in this system. The portable suction pump is a pump specially designed for negative pressure wound therapy use and provides a vacuum at the required therapeutic level. This level is often either −80 mmHg or −125 mmHg, but other vacuum levels can be used depending on the clinical need. The pump may be a battery operated electrical pump, or use a mechanical means to generate the required vacuum levels. The pump 10 is controlled via a microcontroller 14 mounted on a PCB 12 which provides an interface between the power source 11 and the compressor 13. The vacuum levels are monitored by a pressure transducer 17 on the PCB 12, and the compressor 13 is switched on or off by the PCB 12 to control the level of vacuum required. A switch 18 on the PCB 12 turns the pump on and off, and LEDs 15 and a buzzer 16 provide feedback on the status of the pump to the user. A one way valve 19 between the compressor 13 and the outlet 101 ensures that leakage of vacuum through the compressor valves is kept to a minimum. When the pump 10 is switched on, the compressor 13 is turned on until a preset level of vacuum is reached, a typical value for use in negative pressure wound therapy is −80 mmHg, and then the compressor 13 is switched off. If the pressure falls below a preset value, due to leaks in the pneumatic system of from exudate being pulled out of the wound, the compressor 13 will turn back on until the desired level of vacuum is reached, as read by the pressure transducer 17. A connection 101 from the pump 10 allows a flexible tube to be connected between the compressor in the pump and the flexible exudate canister.

The power source 11 may be a battery power source (for example 2 AA lithium batteries), a capacitor power source, or any other kind of portable electrical power.

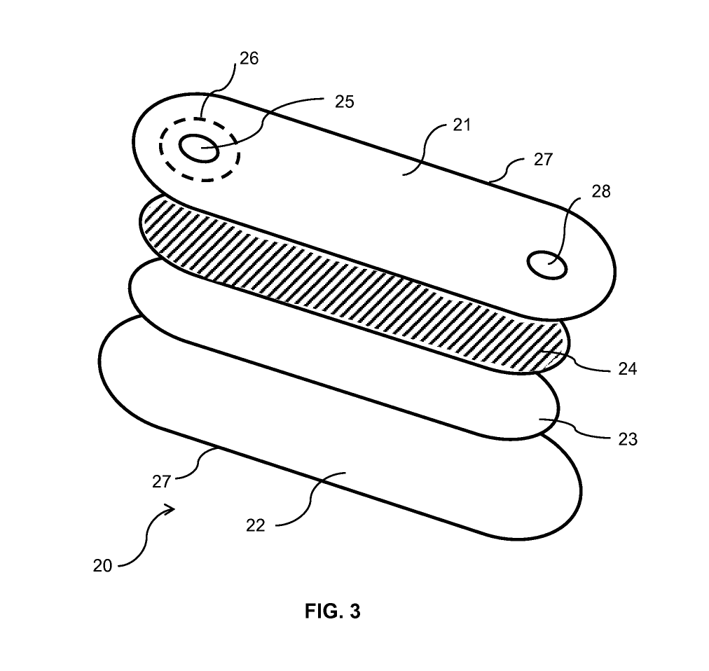

FIG. 3 shows a preferred embodiment of the flexible exudate canister 20. The flexible exudate canister 20 stores the wound exudate that is pulled from the wound by the suction pump. In this figure an exploded perspective view of the flexible exudate canister is shown. The flexible exudate canister 20 is made from a top sheet of thin flexible plastic 21 and a bottom sheet of this thin flexible plastic 22, which may be connected together around their perimeter 27 to make a canister. Typically the material would be PVC or Polyurethane which is high frequency welded around the perimeter, but the material may be any flexible plastic material and the connection method may be any suitable method such as high frequency welding, ultrasonic welding, heat sealing, laser welding, adhesive connections etc.

Sandwiched between the 2 layers of plastic sheeting is a super absorbent polymer in a cellular absorbent matrix 23, the type of which is commonly used in disposable diapers and female hygiene products. This material holds the exudate fluid in its polymer matrix as a gel, preventing spillage and leakage under pressure. The material consists of two sheets an open-cell porous fabric, which may be an air-laid cellulose material, with the super absorbent polymer crystals sandwiched in between them to form a thin sheet. The open-cell porous fabric acts as a wicking agent, drawing the exudate fluid away from the canister sides and into the superabsorbent polymer crystals. The exudate forms a gel with the superabsorbent polymer, expanding in size as it does so.

The super absorbent polymer matrix 23 must have an affinity and molecular bonding for the exudate fluid that is higher than the vacuum applied to the canister, or the flexible sheets 21 and 22 will squeeze the exudate fluid back out of the absorbent matrix. The capacity of the flexible exudate canister will depend on the amount of super absorbent polymer used, and this must be selected depending on the factors of the absorbency per unit area, and the area of the superabsorbent matrix to ensure that the flexible exudate canister will absorb the required volume or weight of exudate. When the superabsorbent polymer matrix 23 has absorbed its designated amount of exudate, any extra exudate sucked into the canister will appear as a liquid on the inner surfaces of the canister and be drawn to the input 25 from the canister to the suction pump. A hydrophobic filter membrane 26 is positioned over the input 25 to stop any fluid from exiting the bag and being drawn into the suction pump.

An air distribution channel structure 24 is placed over the superabsorbent polymer matrix 23 to ensure that the flow of air or exudate fluid from the exudate input 28 to the vacuum input 25 over the superabsorbent polymer matrix 23 is not blocked by the top and bottom layers of plastic sheet 27 and 28 being forced together by the vacuum inside them. This air distribution channel 24 is flexible and can be a woven or knotted nylon mesh material

The outer layer of the bottom thin plastic sheet 22 that forms the flexible exudate canister may be covered with a skin contact adhesive to allow the flexible exudate canister to be suck onto a patient limb as a way of carrying the canister near to the wound site.

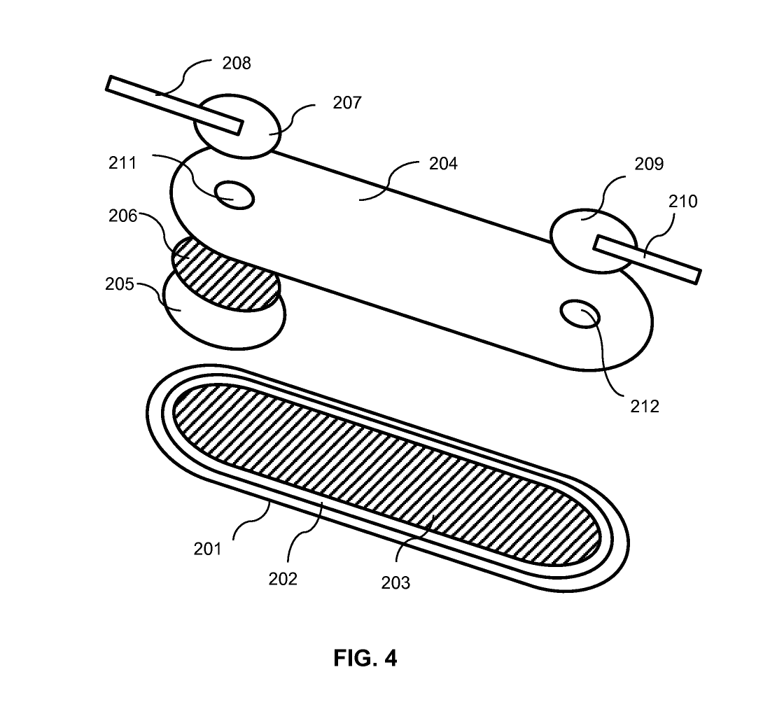

FIG. 4 shows a partial exploded perspective view of a preferred embodiment of the flexible exudate canister 20. In this view the thin plastic base sheet 201, superabsorbent polymer matrix 202 and air distribution layer 203 are all shown in their normal unexploded state, in contact with each other. The first input to the canister 211 is the connection between the suction pump and the canister. This can be a grommet 207 and flexible tube 208 fastened to the top plastic sheet 204, over a hole 211 to allow vacuum access to the inside of the canister. This grommet 207 may typically be PU material high frequency welded to the top sheet 204, but other materials and joining methods such as ultrasonic welding, heat sealing, laser welding or an adhesive connection. Another embodiment is to fasten the tube 208 directly into the canister using a high frequency tube welded joint between the canister outer sheets 204 and the tube 208. Other joining methods could be ultrasonic welding, heat sealing, laser welding, or an adhesive connection.

The second input 212 to the canister the connection between the wound dressing and the canister. This can be a grommet 209 and flexible tube 210 fastened to the top plastic sheet 204, over a hole 212 to allow exudate fluid access to the inside of the canister. The flexible tube 208 and flexible tube 210 should be of different diameters to prevent misconnection in use. This grommet 209 may typically be PU material high frequency welded to the top sheet 204, but other materials and joining methods such as ultrasonic welding, heat sealing, laser welding or an adhesive connection. Other methods may be to fasten the tube 210 directly into the canister using a high frequency tube welded joint between the canister outer sheets 204 and the tube 210. Other joining methods could be ultrasonic welding, heat sealing, laser welding, or an adhesive connection.

A hydrophobic filter membrane 205 is connected on the inside of the top sheet 204, covering the input hole 211. This allows the passage of air into the pump, but not fluid. The filter membrane is attached around its periphery to the top sheet of the canister 204 by high frequency welding, ultrasonic welding, heat sealing, laser welding, or an adhesive connection. The filter membrane is alternatively be attached around its periphery to the bottom of the grommet 207 by high frequency welding, ultrasonic welding, heat sealing, laser welding, or an adhesive connection. In order to ensure there is a sufficient air flow across the filter at all times, an air distribution layer 206 is placed between the top sheet 204 or the grommet body 207 and the filter membrane 205. This material may be the same as used in the canister on the superabsorbent polymer 203 or the air distribution layer may be an air channel shape molded into the face of the grommet that touches the filter disk.

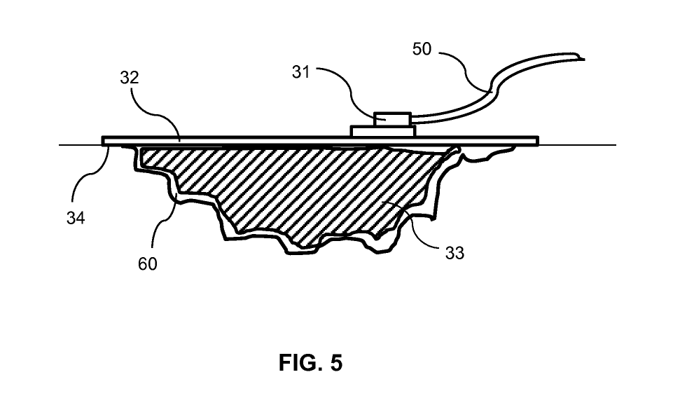

FIG. 5 is a side cutaway view of a preferred embodiment of the wound dressing and wound interface. The wound interface consists of a grommet 31 connected to a flexible tube 50. The grommet 31 may be a polyurethane or similar plastic molded part, with a large flat flange base. The base is covered by a pressure sensitive adhesive with a peel back adhesive backing paper. The grommet has an internal conduit connecting the flat flange to the grommet inlet. The grommet inlet is connected to the flexible tube 50 that comes from the exudate input port of the flexible canister. The connection between the flexible tube and the grommet may be fastened using high frequency welding, ultrasonic welding, heat sealing, laser welding, or an adhesive connection or it may be a mechanical push on connector interface such as a bayonet or barbed connector. The wound interface would normally be provided sterile, in a sterile packing.

The wound dressing has a flexible membrane 32, e.g. a polymeric or elastomeric film, which includes a bio-compatible adhesive on all or part of one side. A preferred embodiment would be to use a standard commercially available transparent film occlusive dressing with a hypoallergenic adhesive (eg Polyskin II from Covidien or Tagerderm from 3M). This would be trimmed to size to suit the particular wound and a small hole cut into it to allow a fluid connection 31 between the wound and the wound interface. The wound interface grommet 31 would have the pressure sensitive adhesive backing removed from the flange and the flange stuck onto the occlusive dressing film 32 such that the connecting hole in the grommet 31 marries up with the hole cut into the occlusive dressing 32 to provide a fluid path. The void in the wound 60 is filled by using a commercially available wound filler 33 (such as Kerlix gauze from Covidien). The wound cover would then have its pressure sensitive backing removed, and the wound cover sealed around the edges of the wound 34 to form an airtight seal.

What is claimed is:

1. A system for applying negative pressure therapy to a wound, comprising:
a portable, electrically powered suction pump, with means of delivering a controlled level of vacuum;
a wound dressing including a occlusive wound cover that is sealable to the skin surrounding the wound;
an exudate canister which is flexible, orientation independent and can conform to the shape of the surface it is mounted to, said canister containing a first input in pneumatic communication with a said vacuum, a second input in fluid communication with said wound dressing and an absorbent material to contain the exudate fluid drawn from said wound;
a fluid interface to connect the exudate canister to said wound cover, said interface in fluid communication with the exudate in the wound;
a conduit connecting the first input of said exudate canister to said wound dressing fluid interface; and
a conduit connecting the second input of said exudate canister to said suction pump.

2. A system of claim 1, wherein said flexible exudate canister comprises of flexible outer surfaces to contain the exudate fluid, an absorbent material between the outer surfaces to hold the exudate fluid in a gel like state, a first input to receive the exudate fluid, a second input connected to a suction pump to apply a vacuum, an air distribution layer and a hydrophobic filter covering the second input to prevent the fluid from exiting the canister to the suction pump

3. A system of claim 1, wherein said flexible exudate canister outer surface is comprised of flexible plastic sheets which are sealed around their periphery.

4. A system of claim 1, wherein said canister outer surfaces are flexible plastic sheets are PVC, polyurethane, or similar flexible plastic sheet material.

5. A system of claim 1, wherein said canister outer surfaces are sealed around their periphery with high frequency welding, ultrasonic welding, heat sealing, laser welding or adhesive bonding.

6. A system of claim 1, wherein said canister contains an absorbent material to contain the exudate fluid, the absorbent material having an affinity for the exudate fluid greater than the negative pressure applied by the suction pump, so that the absorbent material expands when absorbing the fluid and holds the exterior surfaces of the exudate canister apart against the forces of the negative pressure without the need for any internal or external support structure.

7. A system of claim 1, wherein said absorbent material is a super absorbent polymer.

8. A system of claim 1, wherein said absorbent material is a super absorbent polymer surrounded by a wicking layer to draw the exudate fluid into the super absorbent polymer.

9. A system of claim 1, wherein said absorbent material is a super absorbent polymer surrounded by a wicking layer, said wicking layer is a material with an open-cell pore structure, such as an air-laid cellulose sheet.
